(19) United States
(12) Patent Application Publication
(10) Pub. No.: US 2009/0143752 A1
(43) Pub. Date: Jun. 4, 2009

(54) Passive intraocular drug delivery devices and associated methods

(76) Inventors: John W. Higuchi, Salt Lake City, UT (US); S. Kevin Li, Cincinnati, OH (US)

Correspondence Address: THORPE NORTH & WESTERN, LLP., P.O. Box 1219, SANDY, UT, 84091-1219, US

(21) Appl. No.: 11/999,266

(22) Filed: Dec. 3, 2007

(51) Int. Cl. A61F 9/00

(52) U.S. Cl. 604/295

(57) ABSTRACT

The present invention provides methods and devices for the passive delivery of active agents into the eye of a subject. In one aspect, for example, a device for passively delivering an active agent into an eye of a subject may include a housing configured to conform to at least a portion of an eye surface, and a first reservoir component located within the housing and configured to be fluidically coupled to a first ocular surface, where the first reservoir contains an ionized active agent. The device may further include a second reservoir component located within the housing and configured to be fluidically coupled to a second ocular surface located adjacent to the first ocular surface, where the second reservoir component contains a depot forming agent having a charge that is opposite in polarity as compared to the ionized active agent. The ionized active agent and the depot forming agent are configured to form a precipitate when contacted together at room to body temperature and at a pH of between about 4 to about 8 in an aqueous medium. Additionally, the device does not include an electrode.

### FIELD OF THE INVENTION

The present invention relates to systems, methods, and devices for the ocular delivery of an active agent into a subject's eye. Accordingly, the present invention involves the fields of chemistry, pharmaceutical sciences, and medicine, particularly ophthalmology.

### BACKGROUND OF THE INVENTION

Posterior and intermediate eye diseases that require ocular drug delivery to prevent blindness include uveitis, bacterial and fungal endophthalmitis, age-related macular degeneration, viral retinitis, and diabetic retinopathy, among others. For example, the reported incidence of posterior uveitis is more than 100,000 people in the United States. If left untreated, uveitis leads to blindness. It is responsible for about 10 percent of all visual impairment in the U.S. and is the third leading cause of blindness worldwide.

Treatments of intermediate and posterior uveitis are complicated by the inaccessibility of the posterior eye to topically applied medications. Current therapy for intermediate and posterior uveitis requires repeated periocular injections and/or high-dose systemic therapy with corticosteroids. Injections are usually preferred to systemic drug administration because the blood/retinal barrier impedes the passage of most drugs from the systemically circulating blood to the interior of the eye. Additionally, large systemic doses are needed to treat intermediate and posterior uveitis, which often result in systemic toxicities including immunosuppression, adrenal suppression, ulcerogenesis, fluid and electrolyte imbalances, fat redistribution and psychological disorders.

Endophthalmitis affects approximately 10,000 people in the United States each year. Endophthalmitis is typically caused by gram-positive bacteria after ocular surgery or trauma, but it can also be fungal or viral in nature. The current method of treating endophthalmitis is direct injection of antimicrobials into the vitreous. Intravitreal injections are necessary because periocular injections and systemic administration do not deliver efficacious amounts of antibiotics to the target sites in the eye.

Treatments of posterior eye diseases require intravitreal and periocular injections or systemic drug administration. Systemic administration is usually not preferred because of the resulting systemic toxicity as discussed above. While intravitreal and periocular injections are preferable to systemic administration, the half-life of most injected compounds in the vitreous is relatively short, usually on the scale of just a few hours. Therefore, intravitreal injections require frequent administration. The repeated injections can cause pain, discomfort, intraocular pressure increases, intraocular bleeding, increased chances for infection, and the possibility of retinal detachment. The major complication of periocular injections is accidental perforation of the globe, which causes pain, retinal detachment, ocular hypertension, and intraocular hemorrhage. Other possible complications of periocular injections include pain, central retinal artery/vein occlusion, and intraocular pressure increases. Therefore, these methods of ocular drug delivery into the posterior of the eye have significant limitations and major drawbacks. In addition, injections are very poorly accepted by patients. These methods also involve high healthcare cost due to the involvement of skilled and experienced physicians to perform the injections.

Ocular iontophoresis is a noninvasive technique used to deliver compounds of interest into the interior of a patient's eye. In practice, two iontophoretic electrodes are used in order to complete an electrical circuit. In traditional, transscleral iontophoresis, at least one of the electrodes is considered to be an active iontophoretic electrode, while the other may be considered as a return, inactive, or indifferent electrode. The active electrode is typically placed on an eye surface, and the return electrode is typically placed remote from the eye, for example on the earlobe. The compound of interest is transported at the active electrode across the tissue when a current is applied to the electrodes. Compound transport may occur as a result of a direct electrical field effect (e.g., electrophoresis), an indirect electrical field effect (e.g., electroosmosis), electrically induced pore or transport pathway formation (electroporation), or a combination of any of the foregoing.

One potential problem with present ocular iontophoretic methods and devices concerns the actual delivery, or rather, the non-delivery of the drug into the eye tissue. Because the return electrode is located remote from the eye, various conductive pathways may be formed. Such divergence of the electric current will decrease the efficiency of drug delivery to the target sites in the eye, and as a result, much of the drug may be delivered into the tissues surrounding the eye rather than into the eye per se.

Additionally, despite its apparent advantages, iontophoresis is really just a method of limiting the invasiveness of drug delivery into the eye's interior. Once inside the eye, the pharmacokinetics of water soluble compounds are identical to those following intravitreal injections i.e. their half-lives are on the order of a few hours. Therefore, in many cases, traditional iontophoresis must be repeated as frequently as intravitreal injections, leading to patient inconvenience, increased costs, and increased possibility of untoward effects caused by the iontophoretic treatment itself.

The problem of patient compliance may be compounded by the need to receive frequent treatment in a medical facility with high healthcare costs and limited resources and practitioners for treating retinal diseases. Existing ocular iontophoresis systems are not patient-friendly, require multiple parts and assembly to practice, and include clumsy and/or complicated procedures. As such, they require the involvement of experienced healthcare professionals to perform the treatments. Paraprofessional and/or in-home self administration use of such devices are precluded by the technical complexity of many existing iontophoretic devices, as well as the costs of expensive dose-controlling equipment. Individuals have a greater tendency to deviate from a medication regimen when required to leave home for medical treatment, particularly when such treatment is frequent.

### SUMMARY OF THE INVENTION

Accordingly, the present invention provides methods and devices for the passive delivery of active agents into the eye of a subject. In one aspect, for example, a device for passively delivering an active agent into an eye of a subject is provided. Such a device may include a housing configured to conform to at least a portion of an eye surface, and a first reservoir component located within the housing and configured to be fluidically coupled to a first ocular surface, where the first reservoir contains an ionized active agent. The device may further include a second reservoir component located within the housing and configured to be fluidically coupled to a second ocular surface located adjacent to the first ocular surface, where the second reservoir component contains a depot forming agent having a charge that is opposite in polarity as compared to the ionized active agent. The ionized active agent and the depot forming agent are configured to form a precipitate when contacted together at room to body temperature and at a pH of between about 4 to about 8 in an aqueous medium. Additionally, the device does not include an electrode.

A variety of housing configurations are contemplated, and any type of housing that allows the passive delivery of an active agent into the eye of a subject as embodied herein is considered to be within the present scope. For example, in one aspect the housing may be an ocular lens-shaped housing having an ocular contact surface configured to conform to the eye surface. In another aspect, the housing may be configured as a scleral lens-shaped housing. Such a scleral lens-shaped housing may further include a variety of configurations. For example, in one aspect such a housing may cover substantially all of the cornea of the eye. In another aspect, the scleral lens-shaped housing may be configured to expose at least a portion of the cornea. In yet another aspect, the scleral lens-shaped housing may be configured as an annular ring. In a further aspect, the scleral lens-shaped housing may be configured as a corneal lens-shaped housing.

Additionally, numerous reservoir configurations are contemplated, and may thus vary depending on the particular configuration of the housing, the type of drug being delivered, the particular delivery methodology, etc. For example, in one aspect the first reservoir and the second reservoir may be located in a side-by-side orientation. In another aspect, the first reservoir and the second reservoir may be located in a nested orientation. Such a nested configuration may additionally include configurations where the first reservoir and the second reservoir are annular rings.

In some embodiments it may be useful to provide multiple first and/or second reservoirs in a housing. For example, in one aspect either the first reservoir is a plurality of first reservoirs or second reservoir is a plurality of second reservoirs. In another aspect, first reservoir is a plurality of first reservoirs and the second reservoir is a plurality of second reservoirs. In yet another aspect, the plurality of first reservoirs and the plurality of second reservoirs may be positioned circumferentially around the housing. In a further aspect, such circumferentially positioned pluralities of first and second reservoirs may be positioned around the housing in an alternating pattern.

In another aspect of the present invention, a device for passively delivering an active agent into an eye of a subject is provided. Such a device may include a housing configured to conform to at least a portion of an eye surface, and a first reservoir component located within the housing and configured to be fluidically coupled to a first ocular surface, where the first reservoir is configured to contain an active agent and wherein the first reservoir component is not associated with an electrode. The device may further include a second reservoir component located within the housing and configured to be fluidically coupled to a second ocular surface located adjacent to the first ocular surface, where the second reservoir is configured to contain a depot forming agent and wherein the second reservoir component is not associated with an electrode.

In yet another aspect, a system for passively delivering an active agent into an eye of a subject is provided. Such a system may include a housing configured to conform to at least a portion of an eye surface, wherein the housing is not associated with an electrode, and an active agent reservoir located within the housing and configured to be fluidically coupled to a first ocular surface, and wherein the active agent reservoir contains an ionized active agent. The system may further include a depot forming agent having a charge that is opposite in polarity as compared to the ionized active agent, wherein the ionized active agent and the depot forming agent are configured to form a precipitate when contacted together at room to body temperature and at a pH of between about 4 to about 8 in an aqueous medium. The depot forming agent may be located within the housing, or it may be physically distinct therefrom. In one aspect, for example, the depot forming agent may be located within a secondary housing that is physically distinct from the housing. In another aspect, the depot forming agent may be applied to the eye in a form that does not require a housing component.

The present invention additionally provides methods for ocularly delivering active agents via passive techniques. For example, in one aspect a method for forming an active agent depot within the eye of a subject is provided. Such a method may include contacting a housing against an eye surface, where the housing includes a first reservoir component located within the housing and configured to be fluidically coupled to a first ocular surface, where the first reservoir containing an ionized active agent. The housing may further include a second reservoir component located within the housing and configured to be fluidically coupled to a second ocular surface located adjacent to the first ocular surface, where the second reservoir component contains a depot forming agent having a charge that is opposite in polarity as compared to the ionized active agent, and wherein the ionized active agent and the depot forming agent are configured to form a precipitate when contacted together at room to body temperature and at a pH of between about 4 to about 8 in an aqueous medium. The method may further include delivering passively the ionized active agent from the first reservoir component into the eye at a first delivery location, delivering passively the depot forming agent from the second reservoir component into the eye at a second delivery location, and precipitating the ionized active agent and the depot forming agent into an active agent depot at a depot location within the eye that is dependent on the position of the first delivery location relative to the second delivery location.

In another aspect of the present invention, a method for forming an active agent depot within the eye of a subject is provided. Such a method may include contacting a housing against an eye surface, the housing further including an active agent reservoir component located within the housing and configured to be fluidically coupled to a first ocular surface, and wherein the active agent reservoir contains an ionized active agent. The method may further include contacting a depot forming agent with the eye, the depot forming agent having a charge that is opposite in polarity as compared to the ionized active agent, and wherein the ionized active agent and the depot forming agent are configured to form a precipitate when contacted together at room to body temperature and at a pH of between about 4 to about 8 in an aqueous medium. Additionally, the method may include allowing sufficient time for the ionized active agent and the depot forming agent to passively diffuse into the eye and form an active agent depot. While contact of the active agent and the depot forming agent with the eye may be simultaneous, in one aspect the depot forming agent may be contacted with the eye prior to contacting the ocular lens-shaped housing with the eye. In another aspect, the depot forming agent may be contacted with the eye following contacting the ocular lens-shaped housing with the eye.

The particular active agent to be delivered may include a variety of substances depending on the particular treatment to be effected. Such substances may include drugs in various forms, including prodrugs thereof, and sustained release formulations, as required in order to provide convenient and effective non-invasive delivery. Exemplary active agents are enumerated further herein.

### BRIEF DESCRIPTION OF THE DRAWINGS

FIG. 1 is a top view of an ocular device in accordance with an aspect of the present invention.

FIG. 2 is a top view of an ocular device in accordance with another aspect of the present invention.

FIG. 3 is a top view of an ocular device in accordance with yet another aspect of the present invention.

FIG. 4 is a top view of an ocular device in accordance with a further aspect of the present invention.

FIG. 5 is a top view of an ocular device in accordance with another aspect of the present invention.

FIG. 6 is a top view of an ocular device in accordance with another aspect of the present invention.

FIG. 7 is a top view of an ocular device in accordance with yet another aspect of the present invention.

FIG. 8 is a top view of an ocular device in accordance with a further aspect of the present invention.

FIG. 9 is a top view of an ocular device in accordance with a yet a further aspect of the present invention.

### DETAILED DESCRIPTION OF THE INVENTION

Before the present systems and methods for ocular drug delivery are disclosed and described, it is to be understood that this invention is not limited to the particular process steps and materials disclosed herein, but is extended to equivalents thereof, as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and, “the” include plural referents unless the context clearly dictates otherwise. Thus, for example, reference to “a polymer” includes reference to one or more of such polymers, and “an excipient” includes reference to one or more of such excipients.

Definitions

In describing and claiming the present invention, the following terminology will be used in accordance with the definitions set forth below.

As used herein, “formulation” and “composition” may be used interchangeably herein, and refer to a combination of two or more elements, or substances. In some embodiments a composition may include an active agent, an excipient, or a carrier to enhance delivery, depot formation, etc.

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical, and medicinal arts. Examples of drugs useful in the present invention include without limitation, steroids, antibacterials, antivirals, antifungals, antiprotozoals, antimetabolites, immunosuppressive agents, VEGF inhibitors, ICAM inhibitors, antibodies, protein kinase C inhibitors, chemotherapeutic agents, neuroprotective agents, nucleic acid derivatives, aptamers, proteins, enzymes, peptides, and polypeptides.

As used herein “prodrug” refers to a molecule that will convert into a drug (its commonly known pharmacological active form). Prodrugs themselves can also be pharmacologically active, and therefore are also expressly included within the definition of an “active agent” as recited above. For example, dexamethasone phosphate can be classified as a prodrug of dexamethasone, and triamcinolone acetonide phosphate can be classified as a prodrug of triamcinolone acetonide.

As used herein, “effective amount,” and “sufficient amount” may be used interchangeably and refer to an amount of an ingredient which, when included in a composition, is sufficient to achieve an intended compositional or physiological effect. Thus, a “therapeutically effective amount” refers to a non-toxic, but sufficient, amount of an active agent to achieve therapeutic results in treating a condition for which the active agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “sclera” refers to the sclera tissue in the eye or the conjunctiva between the limbus and the fomix on the surface of the eye, which is the white part of the eye. “Sclera” may also be used in referring to other eye tissues.

As used herein, “subject” refers to a mammal that may benefit from the administration of a composition or method as recited herein. Examples of subjects include humans, and may also include other animals such as horses, pigs, cattle, dogs, cats, rabbits, aquatic mammals, etc.

As used herein, “noninvasive” refers to a form of administration that does not rupture or puncture a biological membrane or structure with a mechanical means across which a drug or compound of interest is being delivered. A number of noninvasive delivery mechanisms are well recognized in the transdermal arts such as patches and topical formulations. Many of such formulations may employ a chemical penetration enhancer in order to facilitate non-invasive delivery of the active agent. Additionally, other systems or devices that utilize a non-chemical mechanism for enhancing drug penetration, such as iontophoretic devices are also known. “Minimally invasive” refers to a form of administration that punctures a biological membrane or structure but does not cause excessive discomfort to the subjects and severe adverse effects. Examples of “minimally invasive” drug delivery are microneedle, laser, or heat punctuation in transdermal delivery and periocular injections in ocular delivery.

As used herein, “depot” refers to a temporary mass inside a biological tissue or system, which includes a drug that is released from the mass over a period of time. In some aspects, a depot may be formed by the interaction of an active agent with a depot forming agent, such as a complexing ion which will form an active agent complex that is less soluble than the active agent by itself, and thus precipitate in-vivo.

As used herein, the term “housing” refers to a structure that houses a compound such as an active agent and/or a depot forming agent. In one aspect, the compound may be located within a distinct reservoir component that is located within the housing. In another aspect, the housing material itself may be the reservoir component, as would be the case with a minitab or a hydrogel insert designed to adhere to the eye surface and biodegrade over time.

As used herein, the terms “reservoir” and “reservoir component” may be used interchangeably, and refer to a body or a mass that may contain an active agent, a depot forming agent, or other pharmaceutically useful compound or composition. As such, a reservoir may include any structure that may contain a liquid, a gelatin, a sponge, a semi-solid, a solid or any other form of active agent, depot forming agent, or other compound known to one of ordinary skill in the art. In some cases, an electrode may be considered to be a reservoir.

As used herein, the term “corneal lens” refers to a lens sized to fit approximately over the cornea of the eye.

As used herein, the term “scleral lens” refers to a lens sized to cover and extend beyond the cornea across at least a portion of the sclera of the eye.

As used herein, the term “reacting” refers to any force, change in environmental conditions, presence or encounter of other chemical agent, etc. that alters the active agent. For example, “reacting” between the active agent and the depot forming agent can be physical or chemical interactions.

As used herein, the term “precipitate” refers to anything less than fully solubilized. As such, a precipitate can include not only crystals, but also gels, semi-solids, increased molecular weight, etc.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc.

This same principle applies to ranges reciting only one numerical value. Furthermore, such an interpretation should apply regardless of the breadth of the range or the characteristics being described.

The Invention

The present invention provides devices and associated methods for passively delivering an active agent into the eye of a subject. The inventors have now discovered that an active agent may be effectively delivered into the eye by passive methods. By eliminating power sources, wires, electrodes, etc. that are required for iontophoretic delivery, ocular devices for the administration an active agent can be simplified and greatly reduced in size. Such improvements may increase the ease of use and thus increase subject compliance in the treatment and/or prevention of numerous ocular conditions. Additionally, in some aspects an active agent depot may be formed within the eye to further facilitate the ease of use of such devices and methods.

In one aspect, for example, a device for passively delivering an active agent into an eye of a subject may include a housing configured to conform to at least a portion of an eye surface, and a first reservoir component located within the housing that is configured to be fluidically coupled to a first ocular surface, where the first reservoir contains an ionized active agent. The device may further include a second reservoir component located within the housing that is configured to be fluidically coupled to a second ocular surface located adjacent to the first ocular surface, where the second reservoir component contains a depot forming agent having a charge that is opposite in polarity as compared to the ionized active agent. The ionized active agent and the depot forming agent are configured to form a precipitate when contacted together at room to body temperature and at a pH of between about 4 to about 8 in an aqueous medium. Furthermore, the device does not include an electrode. It should be noted that the device is intended for passive administration of the active agent and the depot forming agent into the eye. Therefore, in some cases devices having an associated electrode that is not used during delivery of the active agent into the eye would also fall within the scope of the present invention.

In another aspect of the present invention, a device for passively delivering an active agent into an eye of a subject may include a housing configured to conform to at least a portion of an eye surface, and a first reservoir component located within the housing and configured to be fluidically coupled to a first ocular surface, where the first reservoir is configured to contain an active agent and where the first reservoir component is not associated with an electrode. The device may further include a second reservoir component located within the housing and configured to be fluidically coupled to a second ocular surface located adjacent to the first ocular surface, where the second reservoir is configured to contain a depot forming agent and where the second reservoir component is also not associated with an electrode.

It is contemplated that the housing may be of any shape having at least a portion that conforms to fit the surface of the eye. In one aspect, the housing may be a single housing containing the first and second reservoirs. In another aspect, the housing may include a first housing containing the first reservoir and a separate second housing containing the second reservoir. One useful example of a housing containing all reservoir components can be configured to substantially conform to the surface of the eye. In one such aspect, the housing may be an ocular lens-shaped housing having an ocular contact surface configured to conform to the eye surface. One specific aspect may include a corneal lens-shaped housing configured to fit at least substantially over the cornea. In another specific aspect, the housing may be a scleral lens-shaped housing, and thus be shaped to fit at least substantially over the scleral tissues of the eyes. In such an example, the ocular lens housing would be configured to extended at least into a cul-de-sac under an eyelid of the eye of the subject. In some cases, a scleral lens-shaped housing may be configured to expose at least a portion of the cornea. One specific example of such a scleral lens-shaped housing may include a housing shaped like an annular ring. Such configuration may be beneficial to allow the subject to see through the cornea while the housing is in position. In one aspect, the ocular lens-shaped housing may be configured such that the reservoir or reservoirs associated therewith are positioned within the lower cul de sac of the eye during use. As the lower eyelid generally exhibits less movement during blinking, such a configuration may assist in maintaining the position of the housing during use.

Various materials are contemplated for use as the housing that may securely hold the various components of the device. It may be additionally beneficial to utilize materials that provide some level of flexibility to avoid damage or irritation to the eye surface. Any material having properties beneficial to the construction of such a device would be considered to be within the scope of the present invention. For example, the housing material may include, without limitation, plastics, metals, composites, Teflon, nylons, polyesters, polyurethanes, polyethylenes, polycarbonates, etc. The housing may additionally be constructed of a material for use as a reservoir. In such cases, it may be useful to construct the housing from a material that biodegrades while associated with the eye as the active agent/depot forming agent is released.

The relative spacing between the first reservoir component and the second reservoir component plays an important role in determining where an active agent depot is localized in the eye upon delivery. As such, in accordance with one aspect of the present invention, the first reservoir component and the second reservoir component may be spaced at a distance that controls the depth and extent of penetration of the active agent depot within the eye. Reservoirs that are spaced further apart on the eye surface allow the active agent and the depot forming agent to diffuse further into the eye before coming into contact and forming a depot as compared to reservoirs that are spaced closer together. Thus, by altering the physical locations of each of the reservoir components the active agent depot can be formed in particular regions of the eye at varying depths. It should be noted, however, that passive administration techniques tend to precipitate drug depots in the eye tissue in regions where the drug reservoir and the depot forming agent reservoir are in close proximity. As such, although the inter-reservoir distance may vary depending on the intended delivery location, passive administration techniques may optimally form drug depots when reservoirs are located close together. In one aspect of the present invention, for example, the inter-reservoir distance may be less than about 5.0 mm. In yet another aspect, the inter-reservoir distance may be less than about 2.0 mm. In a further aspect, the inter-reservoir distance may be from about 0.3 mm to about 1.5 mm.

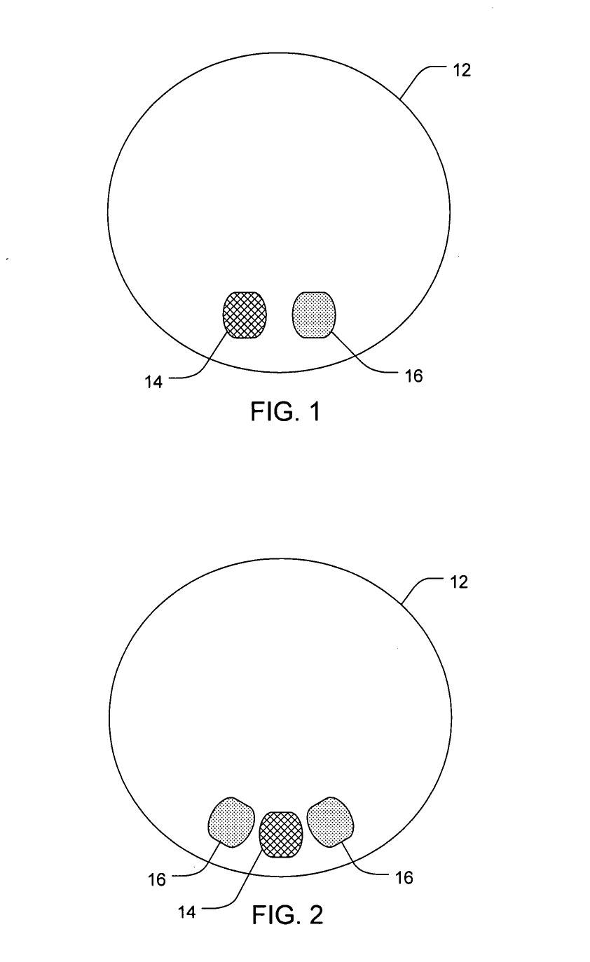

Additionally, the orientation, spacing, and number of the reservoir components on the surface of the eye may be configured in a variety of ways depending on the intended delivery location of the active agent depot. As is shown in FIG. 1, for example, a housing 10 may include a first or active agent reservoir component 14 and a second or depot forming agent reservoir component 16 associated closely therewith. The close spatial association allows the active agent and the depot forming agent to interact within the eye tissues upon passive delivery to precipitate a drug depot. Additionally, utilizing multiple first and/or second reservoir components may increase the effectiveness of delivery of the active agent and the subsequent formation of the active agent depot. In one aspect, for example, the first reservoir may be a plurality of first reservoirs either containing the same active agent or containing different active agents. In another aspect, the second reservoir may be a plurality of second reservoirs either containing the same depot forming agent or containing different depot forming agents. In yet another aspect, the first reservoir may be a plurality of first reservoirs either containing the same active agent or containing different active agents and the second reservoir may be a plurality of second reservoirs either containing the same depot forming agent or containing different depot forming agents. As is shown in FIG. 2, one aspect may include a housing 12 having a single first reservoir component 14 configured to contain an active agent, and multiple second reservoir components 16 configured to contain one or more depot forming agents.

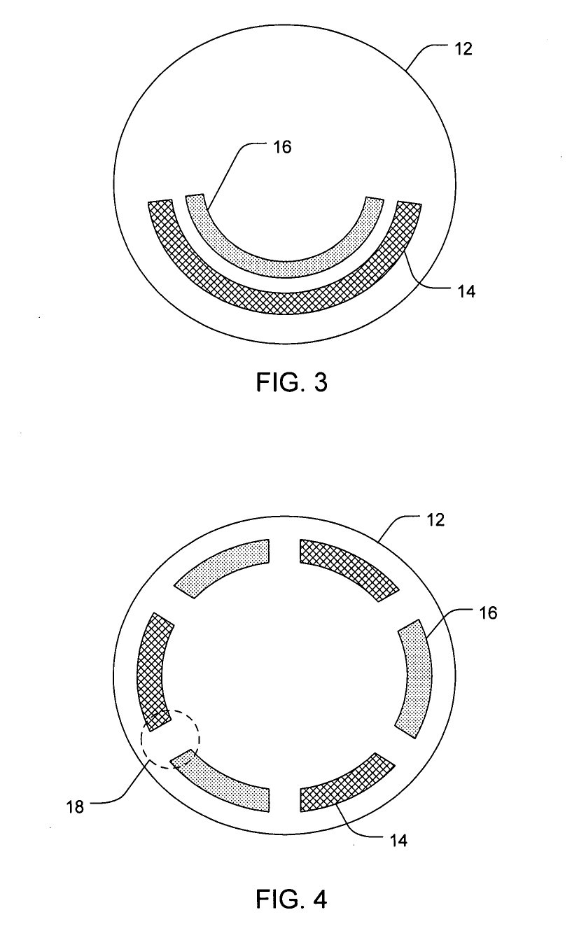

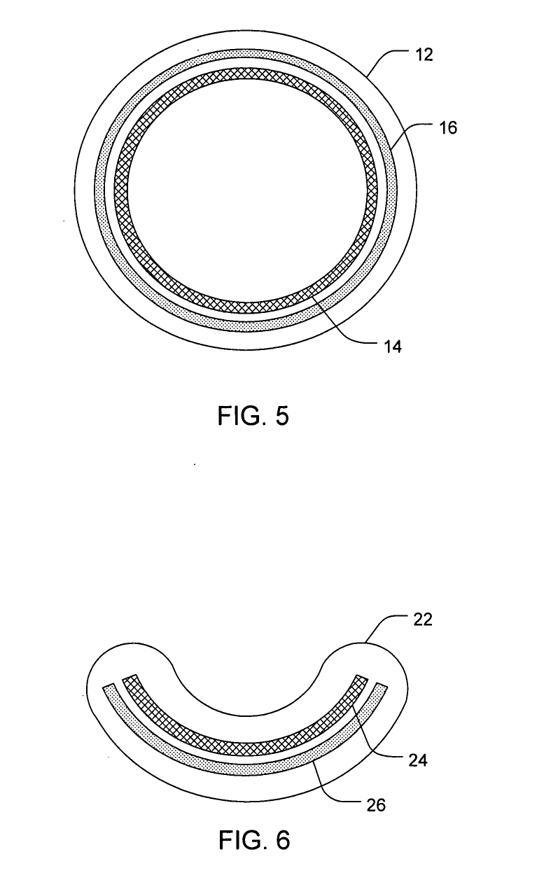

Numerous configurations of reservoirs within a single housing are contemplated, a few of which are described herein. These configurations are merely exemplary, and no limitations to the present scope are intended thereby. In one aspect, for example, the reservoir components may be positioned circumferentially around the housing. Numerous circumferential orientations are contemplated, and may vary depending on the intended location and configuration of the precipitated depot. Additionally, the reservoir components may be configured as annular rings, oriented in a nested configuration, etc. As is shown in FIG. 3, for example, a housing 12 may include a first reservoir component 14 and a second reservoir component 16 oriented in a nested configuration. In such a configuration, drug depot would precipitate within the eye tissue along the boundary between the reservoir components. Such a nested configuration is additionally not limited by the number of first and second reservoir components. In another example, as is shown in FIG. 4, a housing 12 may include a plurality of first reservoir components 14 and a plurality of second reservoir components 16 oriented circumferentially around the housing 12. In one specific aspect the first and second reservoir components may be positioned in an alternating configuration, as is shown in FIG. 4. In such a configuration, drug depots would precipitate within the eye tissue at locations around the circumference wherever a first reservoir component 14 comes into close contact with a second reservoir component 16. One example of such a close contact region is exemplified at 18. In an alternative aspect, as is shown in FIG. 5, a housing 12 may include a first reservoir component 14 and a second reservoir component 16 that are configured as circumferential rings around substantially the entire housing 12. In such a configuration, drug depot would precipitate within the eye tissue in a ring around the eye corresponding to the region between the reservoir components.

It should additionally be noted that a housing may be configured in a variety of shapes and sizes. In one exemplary aspect shown in FIG. 6, for example, a housing 22 may include a first reservoir component 24 and a second reservoir component 26. In such a case, the housing may be configured to cover only a portion of the eye. The configuration shown in FIG. 6 may be useful for insertion into a cul-de-sac of the eye.

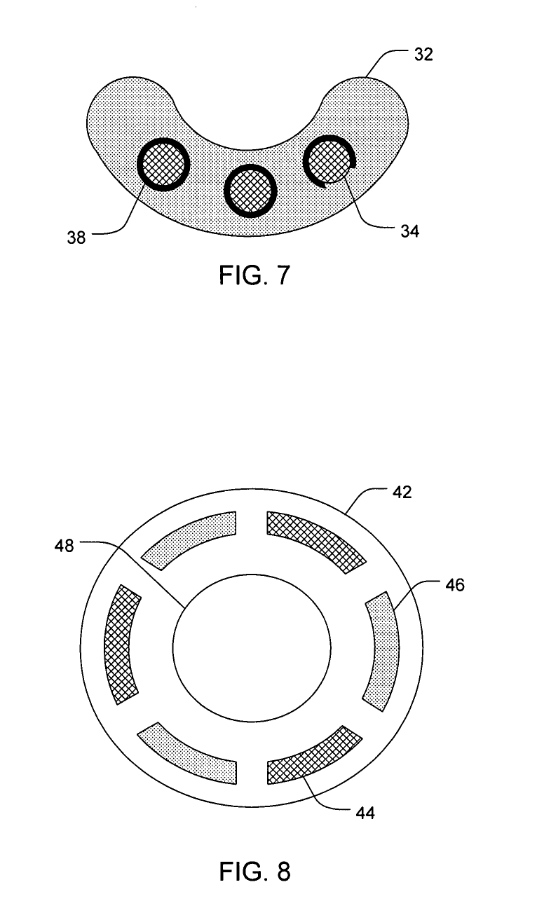

In another aspect, as is shown in FIG. 7, the drug delivery device may be configured to maximize the precipitation of a drug depot in specific locations within the eye tissue. In this case, the housing 32 is configured to contain a depot forming agent substantially throughout the housing material, and thus would function as the second reservoir component. One or more first reservoir components 34 are located within the housing at desired locations for drug depot formation. A barrier element 38 may be used to separate the reservoirs and thus the agents until they have been delivered into the eye, as is discussed more fully below. In such a configuration, as the active agent passively moves into the eye tissue, depot forming agent surrounds the active agent on all sides, causing depot formation substantially directly beneath the first reservoir components 34.

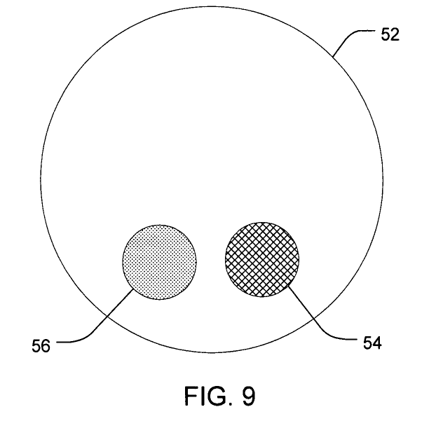

In yet another embodiment, as is shown in FIG. 8, the housing 42 may be configured to expose at least a portion of the cornea of the eye during use. In this particular case, the housing 42 is configured as an annular ring having an open portion 48 through which the cornea is exposed during use. Numerous configurations for the first reservoir component 44 and the second reservoir component 46 are contemplated, as for example, a circumferential orientation as shown in FIG. 8. Allowing exposure of the cornea during use may allow the subject to see beyond the device, thus reducing anxiety that may be associated with drug delivery. In a further aspect, as is shown in FIG. 9, a housing 52 may be configured as a corneal lens-shaped housing having a first reservoir component 54 and a second reservoir component 56.

The reservoirs according to aspects of the present invention are designed to hold an active agent or a depot forming agent to be delivered prior to administration through the eye tissues of a subject. Reservoirs become fluidically coupled to the surface of the eye during use so that the compound contained therein may diffuse passively through the eye tissues. In one aspect, a reservoir may be a distinct compartment within the housing having a lumen for holding an active agent or a depot forming agent. In some aspects, the lumen of the reservoir may contain a matrix to hold the compound to be delivered, such as a sponge, hydrogel, fibrous material, etc. Additionally, such a reservoir may contain at least one access port to allow the reservoir to be filled, either prior to use or while in contact with the body surface of the subject. The later configuration may allow the reservoir to be filled during use as the compound within is depleted. In another aspect, a reservoir may be filled during the manufacture of the device with an active agent or depot forming agent.

In another aspect of the present invention, a housing may include a barrier located between the first reservoir component and the second reservoir component along the housing surface facing the eye to preclude interaction between the active agent and the depot forming agent at the surface of the eye. Such a barrier may also prevent cross-contamination between the first reservoir component and the second reservoir component. The barrier element is thus configured to isolate the first reservoir component and the second reservoir component at the surface of the eye. The barrier element may be located primarily between the reservoirs, or it may be configured to surround each of the reservoirs at the surface of the eye. The barrier element may be constructed of any material known that is capable of forming a barrier. The barrier element material may be the same material as the reservoir and/or the device housing, or it may be a different material selected for its physical or chemical properties. The barrier element may also be physically coupled to the reservoir component, or it may be a protruding portion of the device housing, and thus be continuous with the housing or reservoir. In those aspects where the barrier element is not continuous with the housing or reservoir, the barrier element may be comprised of a material that is either the same or different from either the device housing or the reservoir component material. Non-limiting examples of barrier element materials may include plastics, composites, nylons, polyesters, polyurethanes, polyethylenes, polycarbonates, silicones, etc.

In another aspect, the housing material itself may be the reservoir. In such cases the active agent and/or depot forming agent is released from the housing material once associated with the eye. Such a housing material may be removed from the eye following delivery of the compound, or it may be configured to biodegrade while associated with the eye. Specific examples of the latter may include minitabs, inserts, pastes, hydrogels, punctal plugs, etc. If the active agent and the depot forming agent are located within such a housing material, it is important to keep these compounds separate until diffusion into the eye tissue has occurred. This may be accomplished by including a separate reservoir within the housing material for one of the components, as is shown in FIG. 7. Additionally, a barrier may be utilized to maintain isolation between a housing material that contains an active agent and a housing material that contains a depot forming agent. In another aspect, separate housing elements may be utilized where the housing materials acts as reservoirs. Such housings may be applied to the surface of the eye separately. This may entail simultaneous application of both housing structures to the eye, or serial applications where a second housing is applied following removal of the first housing. For example, a housing material containing a depot forming agent may be applied to the eye to allow the diffusion of the depot forming agent into the eye. The depot forming agent housing may then be removed and replaced by a housing containing an active agent. The active agent will diffuse into the eye tissue and interact with the depot forming agent that was previously administered to form a drug depot. Alternatively, the active agent may be administered prior to the depot forming agent. Such a delivery paradigm may be useful, particularly in those circumstances where an active agent tends to diffuse back out of the eye following delivery. By applying the depot forming agent housing to the region where the active agent has been administered, such “back-diffusion” of the active agent may be minimized by the sequential influx of the depot forming agent into that same region. Additionally, such “back-diffusion” of the active agent may be minimized by the physical presence of the depot forming agent housing.

Various reservoir materials are known to those skilled in the art, and all are considered to be within the scope of the present invention. Additionally, the active agent or depot forming agent may be included in the reservoir in any form, including, without limitation, a liquid, a sponge, a gelatinous, a semi-solid, or a solid form.

In addition to active delivery techniques, aspects of the present invention also provide ocular delivery devices and associated methods that utilize passive techniques to delivery the active agent into the eye. Thus an ocular lens device may be positioned on the eye for a sufficient period of time to allow an active agent located therein to diffuse from the device and into the eye. The various techniques for maintaining alignment between the reservoir surface contact area and the delivery area may thus facilitate the immobilization of the ocular lens device with respect to the eye surface for the longer durations that may be required to passively deliver an active agent to the eye.

In another aspect of the present invention, various systems are also provided. For example, in one aspect a system for passively delivering an active agent into an eye of a subject may include a housing configured to conform to at least a portion of an eye surface, where the housing is not associated with an electrode, and an active agent reservoir located within the housing and configured to be fluidically coupled to a first ocular surface, where the active agent reservoir further contains an ionized active agent. The system may further include a depot forming agent having a charge that is opposite in polarity as compared to the ionized active agent, wherein the ionized active agent and the depot forming agent are configured to form a precipitate when contacted together at room to body temperature and at a pH of between about 4 to about 8 in an aqueous medium. In one aspect, the depot forming agent may be applied to the eye separately from the delivery by the housing. For example, the depot forming agent may be applied as drops or some other topical application technique before or after delivery of the active agent. In another aspect, the depot forming agent may be located within a secondary housing that is physically distinct from the housing containing the active agent.

In another aspect of the present invention, various methods are also provided. For example, in one aspect a method for forming an active agent depot within the eye of a subject may include contacting a housing against an eye surface, where the housing further includes a first reservoir component located within the housing and configured to be fluidically coupled to a first ocular surface, the first reservoir containing an ionized active agent, and a second reservoir component located within the housing and configured to be fluidically coupled to a second ocular surface located adjacent to the first ocular surface, the second reservoir component containing a depot forming agent having a charge that is opposite in polarity as compared to the ionized active agent, wherein the ionized active agent and the depot forming agent are configured to form a precipitate when contacted together at room to body temperature and at a pH of between about 4 to about 8 in an aqueous medium. Following contacting the housing to the eye, the ionized active agent is delivered passively from the first reservoir component into the eye at a first delivery location and the depot forming agent is delivered passively from the second reservoir component into the eye at a second delivery location. The ionized active agent and the depot forming agent are then precipitated into an active agent depot at a depot location within the eye that is dependent on the position of the first delivery location relative to the second delivery location.

Various timing paradigms are contemplated for delivery of the application of the active agent and the depot forming agent into the eye. For example, in one aspect the delivery housing may be contacted to the eye for a time period sufficient to allow the housing to biodegrade. In another aspect, the housing may be contacted to the eye for a time period sufficient to allow at least substantially all of the active agent contained therein to diffuse into the eye. In yet another aspect, the housing may be contacted to the surface of the eye for a time period of less than or equal to about 1 hour. In a further aspect, the housing may be contacted to the surface of the eye for a time period of from about 1 minute to about 30 minutes. In yet a further aspect, the housing may be contacted to the surface of the eye for a time period of from about 30 seconds to about 5 minutes.

In yet another aspect of the present invention, a method for forming an active agent depot within the eye of a subject may include contacting a housing against an eye surface, where the housing further includes an active agent reservoir component located within the housing that is configured to be fluidically coupled to a first ocular surface, and where the active agent reservoir contains an ionized active agent. Additionally, the method may additionally include contacting a depot forming agent with the eye, where the depot forming agent has a charge that is opposite in polarity as compared to the ionized active agent, and wherein the ionized active agent and the depot forming agent are configured to form a precipitate when contacted together at room to body temperature and at a pH of between about 4 to about 8 in an aqueous medium. The method may further include allowing sufficient time for the ionized active agent and the depot forming agent to passively diffuse into the eye and form an active agent depot.

Though numerous conditions would benefit from the methods and devices of the present invention, they are particularly well suited for the treatment of ocular diseases such as direct, combinatory, and adjunctive therapies. This is because of the relatively high permeability of the eye tissues and the large aqueous compartments in the eye. Examples of eye diseases include without limitation, macular edema, age related macular degeneration, anterior, intermediate, and posterior uveitis, HSV retinitis, diabetic retinopathy, bacterial, fungal, or viral endophthalmitis, eye cancers, glioblastomas, glaucoma, and glaucomatous degradation of the optic nerve.

Accordingly, a wide range of active agents may be used in the present invention as will be recognized by those of ordinary skill in the art. In fact, any agent that may be beneficial to a subject when administered ocularly may be used. Examples of active agents that may be used in the treatment of various conditions include, without limitation, analeptic agents, analgesic agents, anesthetic agents, antiasthmatic agents, antiarthritic agents, anticancer agents, anticholinergic agents, anticonvulsant agents, antidepressant agents, antidiabetic agents, antidiarrheal agents, antiemetic agents, antihelminthic agents, antihistamines, antihyperlipidemic agents, antihypertensive agents, anti-infective agents, antiinflammatory agents, antimigraine agents, antineoplastic agents, antiparkinsonism drugs, antipruritic agents, antipsychotic agents, antipyretic agents, antispasmodic agents, antitubercular agents, antiulcer agents, antiviral agents, anxiolytic agents, appetite suppressants, attention deficit disorder and attention deficit hyperactivity disorder drugs, cardiovascular agents including calcium channel blockers, antianginal agents, central nervous system (“CNS”) agents, beta-blockers and antiarrhythmic agents, central nervous system stimulants, diuretics, genetic materials, hormonolytics, hypnotics, hypoglycemic agents, immunosuppressive agents, muscle relaxants, narcotic antagonists, nicotine, nutritional agents, parasympatholytics, peptide drugs, psychostimulants, sedatives, steroids, smoking cessation agents, sympathomimetics, tranquilizers, vasodilators, β-agonists, and tocolytic agents, and mixtures thereof.

Additionally, further examples of active agents may include steroids, aminosteroids, antibacterials, antivirals, antifungals, antiprotozoals, antimetabolites, VEGF inhibitors, ICAM inhibitors, antibodies, protein kinase C inhibitors, chemotherapeutic agents, immunosuppressive agents, neuroprotective agents, analgesic agents, nucleic acid derivatives, aptamers, proteins, enzymes, peptides, polypeptides and mixtures thereof. Specific examples of useful antiviral active agents include acyclovir or derivatives thereof.

Specific examples of active agents may also include hydromorphone, dexamethasone, amikacin, oligonucleotides, F_{ab} peptides, urokinase-derived peptides, urokinase plasminogen activator (uPA)-derived peptides, Urokinase/Urokinase Receptor Systems, PEG-oligonucleotides, salicylate, tropicamide, methotrexate, 5-fluorouracil, squalamine, triamcinolone acetonide, prednisone, betamethasone, diclofenac, combretastatin A4, mycophenolate mofetil, mycophenolic acid, rapamycin, cyclosporine, and prodrugs, metabolites, and mixtures thereof.

Under a number of circumstances, the active agent used may be a prodrug, or in prodrug form. Prodrugs for nearly any desired active agent will be readily recognized by those of ordinary skill in the art. Additionally, prodrugs that are water soluble that metabolize into drugs with a low aqueous solubility may be beneficial. Because the prodrug is water soluble, it is effectively delivered into the eye via passive delivery. The prodrug then converts into the low solubility drug in the eye and the insoluble drug precipitates to form a depot. The drug in depot form within the eye will be slowly released and provide an ocular sustained release condition.

Though any prodrug would be considered to be within the scope of the present invention, examples may include the derivatives of steroids, antibacterials, antivirals, antifungals, antiprotozoals, antimetabolites, VEGF inhibitors, ICAM inhibitors, antibodies, protein kinase C inhibitors, chemotherapeutic agents, immunosuppressive agents, neuroprotective agents, analgesic agents, nucleic acid derivatives, aptamers, proteins, enzymes, peptides, polypeptides, and mixtures thereof. One specific example of a steroid derivative may include triamcinolone acetonide phosphate or other derivatives of triamcinolone acetonide, dexamethasone phosphate, etc. For example, it may be preferable to label a steroid with one or more phosphate, sulfate, or carbonate functional groups, so the prodrug can be effectively delivered into the eye and form a complex with the precipitating ion.

Various methods of providing sustained release, and therefore sustained therapeutic effect, are contemplated, some of which have been discussed herein. Such a sustained release may be due to a property of the active agent, the use of a prodrug, the use of a sustained release depot, etc. In one aspect, a sustained release depot may be formed in the eye tissue by the reaction of an active agent with a secondary substance such as a depot forming agent following delivery of the active agent to the subject. The depot forming agent and the active agent do not interact with one another until the active agent is delivered into the subject. As such, in most cases the active agent and the depot forming agent will be separated until both are located in-vivo. Thus an in-vivo reaction between the active agent and the depot forming agent will cause the active agent or a derivative thereof to form a depot. In one aspect such a depot forming mechanism may be a change in the solubility of the active agent or a derivative of the active agent, thus causing precipitation and subsequent depot formation. This depot of active agent complex is then able to deliver a therapeutic compound to the biological system over time.

As a sustained release mechanism, it will be recognized that the depot formulation of the present invention generally has an in-vivo solubility that is lower than that of the active agent by itself. In this way, as the active agent dissolves out of the depot over time, a sustained therapeutic effect may be obtained. Further, since the active agent in the depot is unable to have a therapeutic effect until released therefrom, the solubility properties of the depot limit potential toxicity or overdose concerns that would normally arise when delivering a sufficient amount of drug to last over a prolonged period. Further details on such depot administration and depot agents can be found in U.S. patent application Ser. Nos. 11/238,144 and 11/238,104, both filed on Sep. 27, 2005, both of which are incorporated herein by reference.

Various reactions are contemplated that result in a sustained release depot being formed. The reaction between the active agent and the depot forming agent may include an ionic association. Accordingly, in one aspect the depot forming agent can have at least one opposite charge to at least one of the charged groups on the active agent. In another aspect, the depot forming agent can have more than one charge and will be capable of being juxtaposed with more than one charge on the active agent. In yet another aspect, the charges on the depot forming agent can be polyvalent, allowing more than one active agent ion to enter the depot complex. This may allow stronger associations between depot forming agents and active agents, thereby forming a depot complex with a significantly lower solubility constant, Ksp, than the active agent delivered, and thus increasing the potential duration of therapy. In one aspect, therefore, the depot forming agent may be an ion. Examples of useful depot forming agents include without limitation, Ca2+, Sn2+, Fe2+, Fe3+ Mn2+, Mg2+, Zn2+, NH4+, ions of the transition metals in the periodic tables, PO43-, CO32-, SO42-, organic cations, organic anions, polyvalent metals, chelation agents, and ionic pharmaceutical excipients generally used in the pharmaceutical industry or known to the people skilled in the art.

The ratio of depot forming agent to active agent could be one to one. However, in the case of polyvalent depot forming agents, more than one active agent may complex with the same depot forming agent to form a depot complex. In one aspect, the depot complex may have a ratio of depot forming agent to active agent of from about 1:1 to about 1:4. In another aspect, the ratio may be about 1:1. In a further aspect, the ratio may be about 1:2. In yet another aspect, the ratio may be about 1:3. In yet a further aspect, the ratio may be about 1:4. In one more aspect, the ratio of depot forming agent to active agent may be from about 4:1 to about 1:4.

Two or more depot forming agents can be used at the same time to form the sustained release depot. With multiple depot forming agents, the concentration of each depot forming agent for precipitating the same total amount of active agent in the eye can be reduced. This effectively reduces the concentrations of the depot forming agent in the eye during and after delivery, so the depot forming agent concentrations are always below the levels that may cause adverse effects in the eye. The use of multiple depot forming agents also provides other advantages. For example, sustained release can be further controlled by using multiple depot forming agents that have different depot complex-Ksp values.

Other examples of depot forming agents may include, without limitation, catalysts, polymerization initiators, pegylating agents, solvents, pH, thermal, or ionic strength sensitive polymers, active agents used in the treatment of eye diseases, aminosteroids such as squalamine, derivatives of triamcinolone acetonide, enzymes, and combinations and mixtures thereof.

Typically, the depot forming agent is non-toxic in the body and the eye. The solid depot complex should be non-toxic and should not cause significant side effects in the eye. The formation of the depot complex should also occur rapidly to prevent pre-complexation clearance of the active agent or depot forming agent from the vitreous. Additionally, in one aspect the depot forming agent and active agent concentrations required for the nucleation of the depot should be low, however in some cases a wide range of concentrations required for nucleation may be utilized in order to optimize the precipitate, and such would be considered to be within the scope of the present invention. The depot complex has decreased solubility as compared to the non-complexed active agent, and as such has reduced clearance from the eye in its complex form as compared to the active agent alone. In some aspects, the depot complex may have a low solubility. As such, the clearance of the depot forming agent and the active agent in the eye should be relatively slow compared with the precipitation process to allow the completion of depot formation.

In another aspect, the reaction process can result in depot complexes in the form of a gel or aggregation, and may alternatively be crystalline or amorphous in form. In this case, the depot complex should not create any unwanted side effects in the eye. For example, in one specific aspect the depot may be a gel created by a complex of an active agent such as dexamethasone sodium phosphate and a depot forming agent such as Ca2+ ion. In some aspects, the particulate size within the depot may be controlled or adjusted so as to determine the release rate of the drug. Additionally, in yet another aspect, the reaction process may be a result of the cleavage of a portion of the active agent, thus lowering the aqueous solubility of the active agent. One example of such a process may include the enzymatic cleavage of the active agent. As such, the depot forming agent would be the enzyme.

It is also contemplated that reservoirs containing additional compounds or secondary substances may be incorporated into the passive delivery devices according to aspects of the present invention. The secondary substance may be delivered in a similar or in a different manner compared to the active agent. A variety of secondary substances may be included in a secondary reservoir component. For example, in one aspect the secondary substance may include a secondary active agent, a vasoconstrictor agent, or a combination thereof.

In some cases, for example, ocular treatment may be hampered by the in-vivo movement/clearance of the active agent in the eye. It is therefore contemplated that various means for restricting or slowing such movement may improve the effectiveness of the active agent therapy. In one aspect, the in-vivo movement may be restricted by constriction of the blood vessels exiting an area in which the active agent is being delivered or precipitated. Such constriction may be induced by the administration of a secondary substance such as a vasoconstricting agent. Specific non-limiting examples of vasoconstricting agents may include a-agonists such as naphazoline, and tetrahydrozoline, sympathomimetics such as phenylethylamine, epinephrine, norepinephrine, dopamine, dobutamine, colterol, ethylnorepinephrine, isoproterenol, isoetharine, metaproterenol, terbutaline, metearaminol, phenylephrine, tyramine, hydroxyamphetamine, ritrodrine, prenalterol, methoxyamine, albuterol, amphetamine, methamphetamine, benzphetamine, ephedrine, phenylpropanolamine, methentermine, oxymetazoline, phentermine, fenfluramine, propylhexedrine, diethylpropion, phenmetrazine, and phendimetrazine. In one specific aspect, the vasoconstricting agent is oxymetazoline. Vasocontricting agents can be administered either before or concurrently with the administration of the active agent. Though administration of the vasoconstrictor may occur following administration of the active agent, the results may be less effective than prior or concurrent administration.

### EXAMPLES

### Example 1

A 100 microliter/30 G needle Hamilton syringe is used to administer a sub conjunctiva injection (n=2) of 20 microliters of 0.1M DSP at pH 7 into an eye of a New Zealand White Rabbit. The initial dose of DSP is 1.04 mg. After 4 hours from the time of the injection, the rabbit is euthanized and the eye is dissected and analyzed for the presence of DSP. The process is repeated for a total of 2 trials. The results are summarized in Table 1. About 23 micrograms of the approximate 1.04 mg subconjunctival injection of DSP are recovered from the whole eye after 4 hours. This illustrates the clearance effects of the transscleral pathway; in this case about 97.8 percent of the drug cleared the eye within four hours.

### Example 2

A six chambered annular lens-shaped housing is used to passively administer dexamethasone sodium phosphate (DSP) into the eye for immediate release. Chambers 1, 3, and 5 each contain a 2 mm Avalon Sponge hydrated with 25 μl of hydrogel and are further loaded with 6.47 mgs of 0.5 M DSP at pH 7 in each chamber. The housing is coupled to an eye of a New Zealand White Rabbit and depressurized with 0.2 cc of suction. The housing is maintained on the eye for 20 minutes. After 4 hours from the time the housing was applied to the eye, the rabbit is euthanized and the eye is dissected and analyzed for the presence of DSP. The process is repeated for a total of 4 trials. The results are summarized in Table 1.

### Example 3

A six chambered annular lens-shaped housing is used to passively administer DSP and a depot forming agent into the eye for sustained release. Each chamber of the housing contains a 2 mm Avalon Sponge hydrated with 25 μl of hydrogel. Chambers 1, 3, and 5 are further loaded with 6.47 mgs of 0.5 M dexamethasone sodium phosphate (DSP) at pH 7 in each chamber. Chambers 2, 4, and 6 are further loaded with 1.0 M CaCl₂. The housing is coupled to an eye of a New Zealand White Rabbit and depressurized with 0.2 cc of suction. The housing is maintained on the eye for 20 minutes. After 4 hours from the time the housing was applied to the eye, the rabbit is euthanized and the eye is dissected and analyzed for the presence of DSP. The process is repeated for a total of 4 trials. The results are summarized in Table 1.

TABLE 1

| | | | Example 3 |
|---|---|---|---|
| | Example 1 | Example 2 | DSP-CaCl₂ |
| | DSP Total Drug | DSP Total Drug | Total Drug |
| | (micrograms) | (micrograms) | (micrograms) |
| Sclera | 10.8 ± 7.7 | 4.3 ± 1.0 | 46.7 ± 23.5 |
| Retina/Choroid | 0.8 ± 0.2 | 1.1 ± 0.5 | 3.5 ± 1.9 |
| Conjunctiva | 10.8 ± 8.7 | 6.2 ± 0.8 | 101.5 ± 24.1 |
| Whole Eye | 23.1 ± 0.6 | 14.9 ± 1.1 | 168.7 ± 52.3 |

### Example 4

A delivery housing having two concentric rings is used to deliver triamcinolone acetonide phosphate (TAP). 35.5 mgs of 0.5 M TAP is loaded into the inner ring of the housing. The outer ring is loaded with 1.0 M CaCl₂. The housing is coupled to an eye of a New Zealand White Rabbit. The housing is maintained on the eye for 20 minutes. After 6 hours from the time the housing was applied to the eye, the rabbit is euthanized and the eye is dissected and analyzed for the presence of TAP. The results are summarized in Table 2.

### Example 5

A delivery housing having 6 side-by-side annular chambers is used to deliver triamcinolone acetonide phosphate (TAP). 5.9 mgs of 0.5 M TAP is loaded into chambers 1, 3, and 5. Chambers 2, 4, and 6 are loaded with 1.0 M ZnCl₂. The housing is coupled to an eye of a New Zealand White Rabbit. The housing is maintained on the eye for 20 minutes. After 6 hours from the time the housing was applied to the eye, the rabbit is euthanized and the eye is dissected and analyzed for the presence of TAP. The results are summarized in Table 2.

TABLE 2

| | Example 4 - TAP-Ca | Example 5 - TAP-Zn |
|---|---|---|
| | Total Drug (micrograms) | Total Drug (micrograms) |
| Sclera | 13.1 | 4.83 |
| Retina/Choroid | 1.394 | 0.62 |
| Conjunctiva | 39.5 | 14.3 |
| Whole Eye | 69.41 | 21.7 |

### Example 6

A delivery housing having two concentric rings is used to deliver mycophenolic acid (MPA). 23.2 mgs of 0.6 M MPA is loaded into the inner ring of the housing. The outer ring is loaded with 1.0 M Zn. The housing is coupled to an eye of a New Zealand White Rabbit. The housing is maintained on the eye for 20 minutes. After 6 hours from the time the housing was applied to the eye, the rabbit is euthanized and the eye is dissected and analyzed for the presence of MPA. The results are summarized in Table 3.

TABLE 3

| | Example 6 - MPA-Zn |
|---|---|
| | Total Drug (micrograms) |
| Sclera | 3.56 |
| Retina/Choroid | 0.99 |
| Conjunctiva | 13.6 |
| Whole Eye | 22.1 |

It should be understood that the above-described arrangements are only illustrative of the application of the principles of the present invention. Numerous modifications and alternative arrangements may be devised by those skilled in the art without departing from the spirit and scope of the present invention. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A device for passively delivering an active agent into an eye of a subject, comprising:
a housing configured to conform to at least a portion of an eye surface;
a first reservoir component located within the housing and configured to be fluidically coupled to a first ocular surface, said first reservoir containing an ionized active agent;
a second reservoir component located within the housing and configured to be fluidically coupled to a second ocular surface located adjacent to the first ocular surface, said second reservoir component containing a depot forming agent having a charge that is opposite in polarity as compared to the ionized active agent;
wherein the ionized active agent and the depot forming agent are configured to form a precipitate when contacted together at room to body temperature and at a pH of between about 4 to about 8 in an aqueous medium; and
wherein the device does not include an electrode.

2. The device of claim 1, wherein the housing is an ocular lens-shaped housing having an ocular contact surface configured to conform to the eye surface.

3. The device of claim 1, wherein the housing is configured to biodegrade during use.

4. The device of claim 1, wherein the first reservoir is a plurality of first reservoirs and the second reservoir is a plurality of second reservoirs.

5. The device of claim 4, wherein the plurality of first reservoirs and the plurality of second reservoirs are positioned circumferentially around the housing.

6. The device of claim 5, wherein the plurality of first reservoirs and the plurality of second reservoirs are positioned circumferentially around the housing in an alternating pattern.

7. The device of claim 1, wherein either the first reservoir is a plurality of first reservoirs or second reservoir is a plurality of second reservoirs.

8. The device of claim 1, wherein the first reservoir and the second reservoir are located in a side-by-side orientation.

9. The device of claim 1, wherein the first reservoir and the second reservoir are located in a nested orientation.

10. The device of claim 9, wherein the first reservoir and the second reservoir are configured as annular rings.

11. The device of claim 9, wherein the first reservoir and the second reservoir are situated within the ocular lens-shaped housing so as to be located in the lower cul de sac of the eye during use.

12. The device of claim 1, wherein the housing is configured as a scleral lens-shaped housing.

13. The device of claim 12, wherein the scleral lens-shaped housing is configured to expose at least a portion of the cornea.

14. The device of claim 13, wherein the scleral lens-shaped housing is configured as an annular ring.

15. The device of claim 1, wherein the housing is configured as a corneal lens-shaped housing.

16. A device for passively delivering an active agent into an eye of a subject, comprising:
a housing configured to conform to at least a portion of an eye surface;
a first reservoir component located within the housing and configured to be fluidically coupled to a first ocular surface, said first reservoir configured to contain an active agent and wherein the first reservoir component is not associated with an electrode; and
a second reservoir component located within the housing and configured to be fluidically coupled to a second ocular surface located adjacent to the first ocular surface, said second reservoir configured to contain a depot forming agent and wherein the second reservoir component is not associated with an electrode.

17. A system for passively delivering an active agent into an eye of a subject, comprising:
a housing configured to conform to at least a portion of an eye surface, wherein the housing is not associated with an electrode;
an active agent reservoir located within the housing and configured to be fluidically coupled to a first ocular surface, said active agent reservoir containing an ionized active agent;
a depot forming agent having a charge that is opposite in polarity as compared to the ionized active agent; and
wherein the ionized active agent and the depot forming agent are configured to form a precipitate when contacted together at room to body temperature and at a pH of between about 4 to about 8 in an aqueous medium.

18. The system of claim 17, wherein the depot forming agent is located within a secondary housing that is physically distinct from the housing.

19. A method for forming an active agent depot within the eye of a subject, comprising:
contacting a housing against an eye surface, the housing further including:
a first reservoir component located within the housing and configured to be fluidically coupled to a first ocular surface, said first reservoir containing an ionized active agent;
a second reservoir component located within the housing and configured to be fluidically coupled to a second ocular surface located adjacent to the first ocular surface, said second reservoir component containing a depot forming agent having a charge that is opposite in polarity as compared to the ionized active agent;
wherein the ionized active agent and the depot forming agent are configured to form a precipitate when contacted together at room to body temperature and at a pH of between about 4 to about 8 in an aqueous medium; and
delivering passively the ionized active agent from the first reservoir component into the eye at a first delivery location;
delivering passively the depot forming agent from the second reservoir component into the eye at a second delivery location; and
precipitating the ionized active agent and the depot forming agent into an active agent depot at a depot location within the eye that is dependent on the position of the first delivery location relative to the second delivery location.

20. The method of claim 19, wherein the housing is contacted to the surface of the eye for a time period of less than or equal to about 1 hour.

21. The method of claim 19, wherein the housing is contacted to the surface of the eye for a time period of from about 1 minute to about 30 minutes.

22. The method of claim 19, wherein the housing is contacted to the surface of the eye for a time period of from about 30 seconds to about 5 minutes.

23. A method for forming an active agent depot within the eye of a subject, comprising:
contacting a housing against an eye surface, the housing further including an active agent reservoir component located within the housing and configured to be fluidically coupled to a first ocular surface, said active agent reservoir containing an ionized active agent;
contacting a depot forming agent with the eye, said depot forming agent having a charge that is opposite in polarity as compared to the ionized active agent, wherein the ionized active agent and the depot forming agent are configured to form a precipitate when contacted together at room to body temperature and at a pH of between about 4 to about 8 in an aqueous medium; and
allowing sufficient time for the ionized active agent and the depot forming agent to passively diffuse into the eye and form an active agent depot.

24. The method of claim 23, wherein the depot forming agent is contacted with the eye prior to contacting the housing with the eye.

25. The method of claim 23, wherein the depot forming agent is contacted with the eye following contacting the housing with the eye.
